(19) United States
(12) Patent Application Publication
(10) Pub. No.: US 2021/0322001 A1
(43) Pub. Date: Oct. 21, 2021

(54) BONE ANCHORING DEVICE

(71) Applicant: KERI MEDICAL SA, Les Acacias (CH)

(72) Inventors: Bernard PRANDI, Lucerne (CH); Julie MOTTET, Pers-Jussy (FR); Damien MOTTE, Annecy (FR); Tanguy MRAOVIC, Baho (FR)

(21) Appl. No.: 17/265,128

(22) PCT Filed: Jul. 9, 2019
(86) PCT No.: PCT/IB2019/055824

§ 371 Date: Feb. 1, 2021

(30) Foreign Application Priority Data
Aug. 3, 2018 (EP) 18187345.6

(51) Int. Cl. A61B 17/04; A61B 17/72

(52) U.S. Cl. CPC A61B 17/0401; A61B 2017/0438; A61B 2017/0414; A61B 17/7291

(57) ABSTRACT

A bone anchoring device includes first and second arms defining a first slot of depth p extending along an axis. The first and second arms respectively bear a first and a second branch extending outside the first slot. The first branch defining second and third slots. In the rest position: the orthogonal projection on the axis of the bottom of each of the second and third slots is in the first slot distanced from the bottom of this slot of at least 10% of p; and the distances d1 separating the second slot bottom from the part of the end of the branch defining it which is the furthest away from it, and d2 separating the third slot bottom from the part of the end of the branch defining it which is the furthest away from it, so each of d1/p and d2/p>0.3.

### CROSS-REFERENCE TO RELATED APPLICATIONS

This application is the U.S. national phase of International Application No. PCT/IB2019/055824 filed Jul. 9, 2019 which designated the U.S. and claims priority to EP 18187345.6 filed Aug. 3, 2018, the entire contents of each of which are hereby incorporated by reference.

### BACKGROUND OF THE INVENTION

### Field of the Invention

The present invention relates to an anchoring device intended to be fixed in a bone during an operation in the field of orthopaedic surgery and in particular to a device such as a suture anchor or an arthrodesis implant.

### Description of the Related Art

Ligament and tendon lesions are among the most common problems encountered in orthopaedic surgery and can affect many different joints. One of the known methods for treating these lesions consists of refixing the damaged tendons or ligaments in place on the bone using an anchor and a suture thread. To this end, the bone is pierced, the anchor is anchored in the bone and a thread which is hooked thereon then allows the damaged tissue to be sutured. There are driven, screwed or “knotted” anchors which may be absorbable or non-absorbable. The invention relates in particular to a suture anchor of the non-absorbable driven type.

During such surgery, it is preferable to preserve as much of the patient's bone as possible and thus to have an anchor which is able to be inserted into a shallow piercing with a small diameter, in particular when the surgery is on a small bone such as a phalanx. Such an anchor must likewise allow high-performance anchoring resistant to traction forces and cyclic forces exerted by the tendons or ligaments.

Furthermore, only the surgeon knows how to evaluate, at the time of operation, the ideal size of the thread necessary to suture the tissues, it is thus advantageous that the suture thread attached to the anchor is accessible and easily interchangeable. However, in numerous anchors of the prior art the suture thread passes through a very small closed-contour opening and so the suture thread is either mounted in the factory, the anchor being delivered to the surgeon with a pre-mounted thread, or is inserted by the surgeon using a thread guide.

Finally, some ligaments have multiple endings and thus require several anchoring points. In order to avoid having to insert several suture anchors, a multi-thread anchor may sometimes prove to be necessary.

International application WO9942064 A1 describes a non-absorbable suture anchor intended to be driven into the bone. For this anchor, anchoring into the bone mass is performed using fins extending radially with respect to the longitudinal axis of the anchor. Since the expansion of the described anchor is relatively small, it does not appear to ensure strong anchoring. Moreover, it appears as though it risks damaging the cortical bone.

Arthrosis, whether primary—when patients do not have clear predisposition—or secondary—when it is the direct consequence of articular diseases, is likewise a common problem in orthopaedics. It particularly affects the joints under the most stress, in particular those in the hand such as the distal interphalangeal (DIP) joint.

To date, no DIP arthroplasty solution has proved to be truly effective. Therefore, arthrodesis is a common operation on this joint because it does not completely handicap the patient and permits indolence. Its aim is to block the articular mobility of the patient and to build a bridge by fusing the bone between the distal phalanx P3 and the middle phalanx P2 in order to eliminate articular arthrosis.

There are different types of implants for DIP arthrodesis: the single-piece or multi-piece intramedullary implant which is inserted via the dorsal route, compression screws which are inserted via the pulpal route and Kirschner orthopaedic pins (K-wires). Since the risks of infection via the pulpal route are higher than via the dorsal route, the intramedullary implant—preferably in one piece—is preferable. Furthermore, passing through the pulp may damage sensitivity of the finger, which of course should be avoided.

This type of implant comprises two bone anchoring zones on either side of a rigid zone. These two anchoring zones are intended to be introduced into sized holes made in the bones to be joined and to be anchored therein in a sufficiently strong manner to ensure good fixing in the bone.

As for the suture anchors, the constraints applying to the anchoring parts of arthrodesis implants, e.g. for DIP arthrodesis, must meet numerous criteria including a small size during insertion (shallow piercing with a small diameter) and strong anchoring to be resistant to the stresses associated with the joint. Forming a small piercing is particularly important in the case of arthrodesis because good preservation of the bone facilitates bone fusion.

French patent application FR 2 913 876 A1 relates to a device for intramedullary arthrodesis comprising two anchoring zones. Each of these anchoring zones comprises two arms defining therebetween a slot extending along an axis and arranged such that the span of the anchoring zone perpendicular to this axis can, from said rest position, be reduced by elastically bringing said two arms closer together, said anchoring zone being intended to be stressed in the folded position for its insertion into a housing formed in the bone, typically a sized centromedullary hole, then to be relaxed to be anchored in the bone under the effect of elastic restoring forces. However, the structure of this implant requires, even in its closed position, a certain diameter for the housing in the bone which it would be beneficial to reduce. Furthermore, the shape of this implant is such that only the ends of the arms come to abut against the cortex and risk damaging it.

### SUMMARY OF THE INVENTION

In a bone anchoring device such as a suture anchor or an arthrodesis implant, intended to be used in orthopaedics, the part to be anchored in the bone must be able to take up a minimum amount of space during insertion thereof to allow a piercing or hole with a minimum span to be formed whilst permitting strong anchoring, able to be resistant to high mechanical stresses. A first object of the invention is to propose a device having these properties.

In the particular case of suture anchors, a second object is likewise to propose an anchoring device allowing easy insertion of the suture thread and the possibility of connected it to several suture threads if required.

To these ends, the invention proposes a bone anchoring device comprising a first and a second arm defining therebetween a first slot of depth p extending along an axis x-x′, said first and second arms respectively bearing a first and a second branch extending outside of said first slot, said first branch being arranged so as to define with the arm which bears it a second slot and said second branch being arranged so as to define with the arm which bears it a third slot, said device being arranged such that, in the rest position:
the orthogonal projection on the axis (x-x′) of the bottom of each of the second (3b; 13b) and third (3c; 13c) slots is located in said first slot (3a; 13a) at a distance from the bottom of this slot of at least 10%, preferably at least 20%, preferably at least 30%, preferably at least 40%, even more preferably at least 50%, of the depth p; and
the distances d1 separating the bottom of said second slot from the part of the end of the branch which defines it which is the furthest away from it, and d2 separating the bottom of said third slot from the part of the end of the branch which defines it which is the furthest away from it, are such that each of the ratios d1/p and d2/p is greater than or equal to 0.3, preferably greater than or equal to 0.4, even more preferably greater than or equal to 0.5;
   said device being able to reduce the span of the assembly comprising said first and second arms and said first and second branches perpendicular to the axis x-x′ from said rest position, on the one hand by elastically bringing each of the branches closer to the arm which bears it, and on the other hand by elastically bringing said first and second arms closer together for the insertion thereof, at least in part, in a bone.

When the span of the assembly comprising said first and second arms and said first and second branches perpendicular to the axis x-x′ is reduced, it is said that this assembly is in the folded position, when it is reduced to the maximum extent, it is said that this assembly is in the extreme folded position.

The assembly comprising the arms and branches of the anchoring device in accordance with the invention is intended to be kept in the folded position or in the extreme folded position by the surgeon, typically using forceps or another instrument, for the insertion thereof in a hole previously formed in a bone. Once said folded assembly is in place in the bone, when the surgeon releases the device from his instrument, it expands in two ways, i.e. both by the arms thereof moving apart and by the branches moving away from the arms, perpendicular to the axis x-x′, to allow strong anchoring of the device in the bone.

The span of the assembly comprising said first and second arms and said first and second branches perpendicular to the axis x-x′ can typically, when these elements are being elastically brought closer together, be reduced by at least 30% with respect to said span in the rest position, i.e. multiplied by a reduction coefficient “k” less than or equal to 0.7. It can preferably be multiplied by a reduction coefficient k less than or equal to 0.6, even more preferably less than 0.55.

A low reduction coefficient k of this span has the advantage of permitting insertion of the assembly comprising the arms and branches of the anchoring device in accordance with the invention into a hole with a minimum span whilst providing said device with strong anchoring, able to be resistant to high mechanical stresses.

Advantageously, the anchoring device in accordance with the invention is designed such that the assembly comprising its arms and branches is able to expand from a first folded position in which its span perpendicular to the axis x-x′ is less than or equal to 0.7, preferably less than or equal to 0.6, even more preferably less than or equal to 0.55, times its span perpendicular to the axis x-x′ in the rest position, to a second position in which its span perpendicular to the axis x-x′ is greater than or equal to 0.75, preferably 0.80, preferably 0.85, preferably 0.90, preferably 0.95, times its span perpendicular to the axis x-x′ in the rest position, even more preferably to its rest position, into the materials of which the compression failure pressure is less than or equal to 10 MPa, preferably less than or equal to 15 MPa, whilst being incapable of expanding even partially into materials of which the compression failure pressure is greater than or equal to 200 MPa, preferably greater than or equal to 100 MPa, preferably greater than or equal to 50 MPa.

These conditions allow the expansion of the assembly comprising the arms and branches of the anchoring device in accordance with the invention to be ensured when located in spongy bone without risking damage to the cortical part of the bone.

Advantageously, in said rest position at least 60%, preferably at least 70%, preferably at least 80%, preferably at least 90%, even more preferably at least 95%, of the orthogonal projection on the axis x-x′ of each of said first and second branches is located in said first slot. In an even more preferred manner, in said rest position the orthogonal projection on the axis x-x′ of each of said first and second branches is located fully or almost fully in said first slot. This feature is used in obtaining a low span reduction coefficient k because it allows a maximum accumulation of span reductions owing on the one hand to each of the branches being elastically brought closer to the arm which bears it and on the other hand said first and second arms being elastically brought closer together.

Advantageously, the bone anchoring device in accordance with the invention is in a single piece.

The bone anchoring device in accordance with the invention is typically made from a biocompatible metal such as titanium or a nickel/titanium alloy. It can also be made for example from a polymer such as PE or PEEK or from polyester fibres. It is preferably made from a nickel/titanium shape memory alloy such as nitinol, typical 55% nickel/45% titanium. Such a material has the advantage of being superelastic.

Preferably, the front and rear surfaces of said first and second arms and said first and second branches are planar and in parallel with a single first plane including the axis x-x′. Even more preferably, the front and rear surfaces of the assembly of the bone anchoring device in accordance with the invention are planar and parallel with said first plane. In this manner it is possible to manufacture a bone anchoring device in accordance with the invention using a simple manufacturing method, by cutting out, typically using a laser, from plates of material, e.g. from plates of nitinol.

Advantageously, the assembly comprising said first and second arms and said first and second branches has an orthogonal plane of symmetry, this plane typically including the axis x-x′. Such a plane of symmetry increases the stability of the anchoring device once in the bone and thus permits better anchoring. Preferably, the assembly of the bone anchoring device in accordance with the invention is symmetrical with respect to said orthogonal plane of symmetry.

A method for placing a bone anchoring device in accordance with the invention comprises at least the following steps:
A. producing a hole extending at least into the spongy part of a bone;
B. reducing the span of the assembly comprising said first and second arms and said first and second branches perpendicular to the axis x-x′ by at least 30%, preferably at least 40%, even more preferably at least 45%, with respect to said span in the rest position, on the one hand by elastically bringing each of the branches closer to the arm which bears it, and on the other hand by elastically bringing said first and second arms closer together;
C. inserting the assembly comprising said first and second arms and said first and second branches into said hole, keeping its span as defined in step B; and
D. ceasing to keep the span as defined in step B such that said assembly expands at least partially into the spongy part of said bone, the span of said assembly perpendicular to the axis x-x′ increasing typically under the effect of elastic restoring forces at least up to 75%, preferably 80%, preferably 85%, preferably 90%, even more preferably 95%, or even 100%, of its span in the rest position.

In a first embodiment of the invention, the bone anchoring device is an orthopaedic suture anchor, said first slot being intended to receive one or more suture threads for suturing a tissue such as a ligament or tendon.

A suture anchor in accordance with the first embodiment of the invention has the advantage of being able to be inserted into a bone in a hole with a small span and of being able to be fixedly anchored in this bone owing to the double expansion of its arms and its branches once in the bone in order to suture damaged tissue. Such a suture anchor likewise has the advantage of allowing insertion of the suture thread(s) by the surgeon, as required, without requiring a thread guide.

Preferably, this bone is selected from small bones such as those in the hand, wrist or foot. Even more preferably, the bone is a phalanx, e.g. a distal phalanx P3.

A method for placing an orthopaedic suture anchor in accordance with the invention typically comprises the following steps:
producing a hole passing all the way through the cortical part of a bone and extending into the spongy part of said bone;
positioning one or more suture threads in the first slot of said suture anchor;
reducing the span of the assembly comprising said first and second arms and said first and second branches perpendicular to the axis x-x′ by at least 30%, preferably at least 40%, even more preferably at least 45%, with respect to said span in the rest position, on the one hand by elastically bringing each of the branches closer to the arm which bears it, and on the other hand by elastically bringing said first and second arms closer together;
inserting the assembly comprising said first and second arms and said first and second branches into said hole, keeping its span as defined in the previously described step;
ceasing to keep said span as in the previously described step such that the assembly expands at least partially into the spongy part of said bone, the span of said assembly perpendicular to the axis x-x′ increasing typically under the effect of elastic restoring forces at least up to 75%, preferably 80%, preferably 85%, preferably 90%, even more preferably 95%, or even 100%, of its span in the rest position; and
suturing a tissue such as a ligament or tendon with the suture thread(s).

In a second embodiment of the invention, the bone anchoring device in accordance with the second embodiment of the invention comprises a first anchoring part comprising said first and second arms and said first and second branches and intended to be anchored in a first bone, and a second anchoring part, made fixedly attached to the first part via a central rigid portion and intended to be anchored in a second bone, the shape of said second anchoring part being able to be identical to or different from that of said first anchoring part. This typically relates to an implant for arthrodesis, preferably an intramedullary implant for distal interphalangeal arthrodesis.

Such a bone anchoring device allows a solid bone bridge to be built between said first and second bone. At least said first anchoring part has the advantage of being able to be inserted into said first bone in a hole with a small span and of being able to be fixedly anchored in this bone owing to the double expansion of its arms and its branches.

Preferably, these two bones are selected from small bones such as those in the hand, wrist or foot. Even more preferably, said first bone is a distal phalanx P3 and said second bone corresponds to the middle phalanx P2 biologically associated with said phalanx P3.

A method for placing an implant for arthrodesis in accordance with the invention typically comprises the following steps:
producing a first hole in the spongy part of a first bone of a joint between two bones to be treated and a second hole in the spongy part of the second bone of said joint;
anchoring the second anchoring part in the second hole; and
anchoring the first anchoring part in the first hole by performing the following steps:
   reducing the span of the assembly comprising said first and second arms and said first and second branches perpendicular to the axis x-x′ by at least 30%, preferably at least 40%, even more preferably at least 45%, with respect to said span in the rest position, on the one hand by elastically bringing each of the branches closer to the arm which bears it, and on the other hand by elastically bringing said first and second arms closer together;
   inserting the assembly comprising said first and second arms and said first and second branches into this hole, keeping its span as defined in the previously described step;
   ceasing to keep the span as in the previously described step such that the assembly expands at least partially into the spongy part of said first bone, the span of said assembly perpendicular to the axis x-x′ increasing typically under the effect of elastic restoring forces at least up to 75%, preferably 80%, preferably 85%, preferably 90%, even more preferably 95%, or even 100%, of its span in the rest position.

The invention likewise relates to a kit comprising a bone anchoring device in accordance with the invention and an instrument able to bear said device and keep it at least in a position in which the span perpendicular to the axis x-x′ of the assembly comprising said first and second arms and said first and second branches is reduced by at least 30% with respect to said span in the rest position.

### BRIEF DESCRIPTION OF THE DRAWINGS

Other features and advantages of the present invention will become clear upon reading the following detailed description given with reference to the attached drawings in which:

FIGS. 1a and 1b show a perspective view of an anchoring device in accordance with a first embodiment of the invention in a first position called “expanded position” and a second position called “extreme folded position” respectively;

FIGS. 2a and 2b show a front view of the anchoring device shown in FIGS. 1a and 1b in said expanded position and said extreme folded position respectively;

FIGS. 3a and 3b show a top view of the device shown in FIGS. 1a and 1b in said expanded position and said extreme folded position respectively;

FIGS. 4a and 4b shows a perspective view of the device shown in FIGS. 1a and 1b in the expanded position with one or two suture threads in place respectively;

FIGS. 5a and 5b show successive steps during insertion of the device shown in FIGS. 1a and 1b through a hole produced in a bone; FIG. 5c illustrates the contours of a hole as shown in FIGS. 5a and 5b in a cross-section of a bone such as a phalanx P3 of a finger of the hand;

FIG. 6a shows a perspective view of an instrument designed to grip and insert the device shown in FIGS. 1a and 1b into the hole shown in FIGS. 5a to 5c; FIGS. 6b and 6c show a transparent view of the device shown in FIGS. 1a and 1b in the expanded and extreme folded positions respectively, within the instrument shown in FIG. 6a. For ease of comprehension, only one part, called active part, of the instrument is shown in these FIGS. 6b and 6c;

FIGS. 7a and 7b show a front view of an anchoring device in accordance with a second embodiment of the invention in a first position called “expanded position” and a second position called “extreme folded position” respectively;

FIGS. 7c and 7d show a top view of the device shown in FIGS. 7a and 7b in said expanded position and said extreme folded position respectively;

FIGS. 8a and 8b show a perspective view and a side view respectively of the anchoring device shown in FIGS. 7a and 7b in said expanded position;

FIGS. 8c and 8d show a perspective view and a side view respectively of a first variant of the device shown in FIGS. 7a and 7b;

FIGS. 8e and 8f show a perspective view and a side view respectively of a second variant of the device shown in FIGS. 7a and 7b;

FIGS. 9a and 9b show successive steps during insertion of the device shown in FIGS. 7a and 7b through a sized hole produced in the bone; FIG. 9c illustrates the contours of a hole as shown in FIGS. 9a and 9b in a cross-section of a bone such as a phalanx P3 of a finger of the hand;

FIG. 10 shows a perspective and transparent view of the device shown in FIGS. 7a and 7b in position in the bone.

### DESCRIPTION OF THE PREFERRED EMBODIMENTS

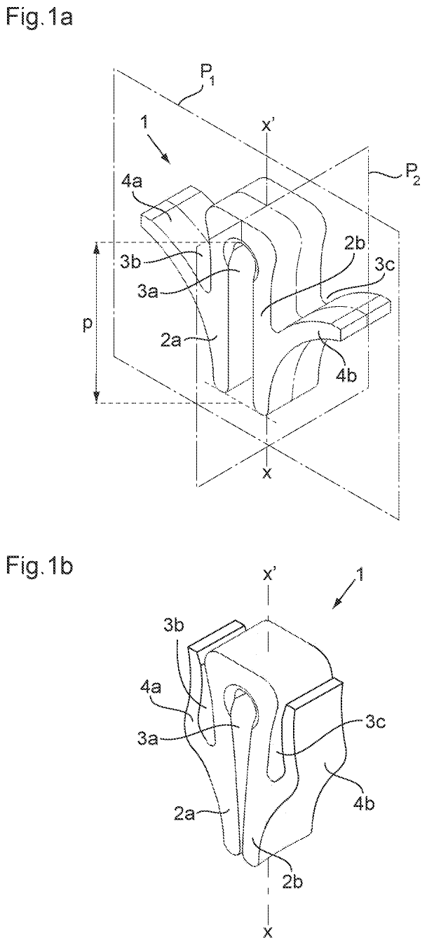

Referring to FIGS. 1a to 5b, a bone anchoring device in accordance with a first embodiment of the invention is a suture anchor 1 intended to be used to refix tissue such as a tendon or ligament on a bone, typically on a distal phalanx (P3) of a finger of the hand.

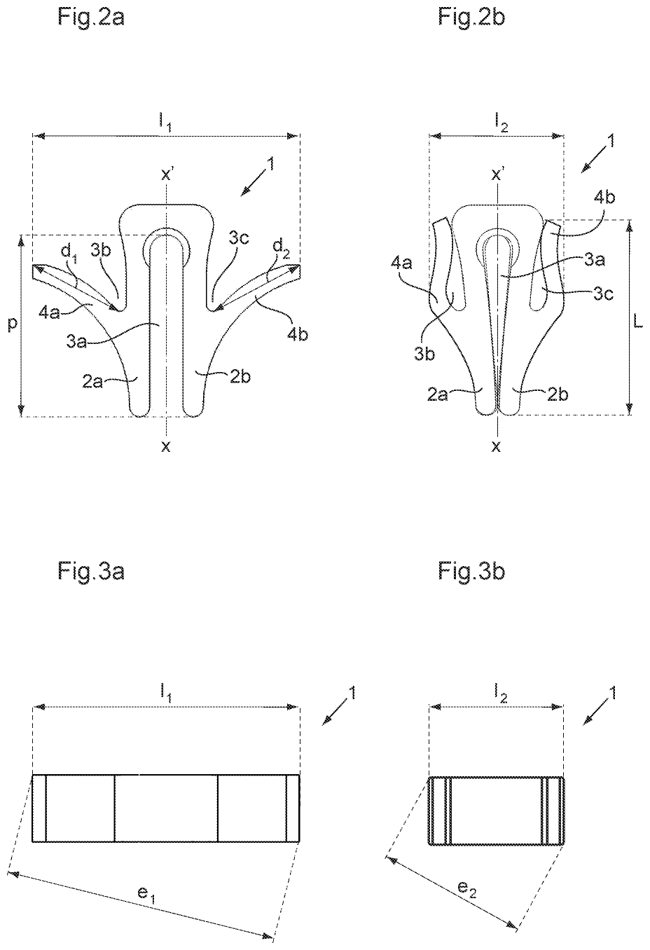

The suture anchor 1 comprises a first 2a and a second 2b arm, typically arranged in a U- or V-shape, defining a first slot 3a therebetween. This first slot 3a has a depth of length p and extends along an axis x-x′, x corresponding to the lower part and x′ corresponding to the upper part of the anchor 1, as shown in FIGS. 1a to 2b.

The first 2a and second 2b arms respectively bear a first 4a and a second 4b branch extending outside of said first slot 3a.

The first branch 4a is arranged so as to define, with the arm 2a which bears it, a second slot 3b. Similarly, the second branch 4b is arranged so as to define, with the arm 2b which bears it, a third slot 3c.

The suture anchor 1 is typically in one piece.

In its rest position, i.e. in the position in which no external force is applied thereon, the anchor 1 is in a position called “expanded”. This rest position is shown in FIGS. 1a, 2a and 3a. In this rest position, the span (maximum or overall) of the anchor 1, perpendicular to the axis x-x′, this span corresponding to the span of the assembly comprising said first 2a and second 2b arms and said first 4a and second 4b branches perpendicular to the axis x-x′, corresponds to a value e1, shown in FIG. 3a.

From this rest position, this span can be reduced on the one hand by bringing the two arms 2a, 2b closer together by elastic deformation and on the other hand by bringing each of the branches 4a, 4b closer to the arm 2a, 2b which bears it, and thus to the axis x-x′, as shown in FIGS. 1b, 2b and 3b.

The position in which the span (maximum or overall) of the anchor 1 perpendicular to the axis x-x′ is minimum will be referred to as the “extreme folded position”. In this position, the ends of the two arms 2a, 2b touch each other such that the contour of the first slot 3a is closed and each of the branches 4a, 4b is folded to the maximum extent against the arm 2a, 2b which bears it. This position is shown in FIGS. 1b, 2b and 3b and corresponds to the ideal position for inserting the anchor 1 into a phalanx P3 of a finger. In this position, the span of the anchor 1, perpendicular to the axis x-x′, this span corresponding to the span of the assembly comprising said first 2a and second 2b arms and said first 4a and second 4b branches perpendicular to the axis x-x′, corresponds to a value e2<e1, shown in FIG. 3b. The reduction coefficient k of this span corresponds to the ratio e2/e1 and is typically less than or equal to 0.7.

The anchor 1 has a span e1, at rest, of 4.1 mm and a span e2, in the extreme folded position, of 2 mm; the reduction coefficient is thus k=0.49. A low reduction coefficient translates into a large expansion potential. Such properties are made possible owing to the use of a superelastic material for manufacturing the anchor 1. The anchor 1 is typically made from a 55% nickel/45% titanium alloy.

The span e1 is slightly greater than the width l1=4 mm of the anchor 1 in the expanded position (FIGS. 2a and 3a). Similarly, the span e2 is slightly greater than the width l2=1.9 mm of the anchor 1 in the extreme folded position (FIGS. 2b and 3b).

The arms 2a, 2b and branches 4a, 4b of the anchor 1 are arranged such that the orthogonal projection on the axis x-x′ of the bottom of each of the second 3b and third 3c slots is located in said first slot 3a at a distance from the bottom of this slot of at least 10%, preferably at least 20%, preferably at least 30%, preferably at least 40%, even more preferably at least 50%, of the depth p. In the example shown in FIGS. 1a to 5b, the orthogonal projection on the axis x-x′ of the bottom of each of the second 3b and third 3c slots is located in said first slot 3a at a distance from the bottom of this slot of about 43%.

Furthermore, the arms 2a, 2b and branches 4a, 4b of the anchor 1 are arranged such that, in the rest position, the orthogonal projection on the axis x-x′ of each of said first 4a and second 4b branches is located fully in said first slot 3a, as shown in FIG. 2a. As a variant, it would be possible for only one part of the length of the orthogonal projection of each of the branches 4a, 4b on the axis x-x′, typically at least 70% of this length, to be located in the first slot 3a in the rest position.

The length of the branches 4a, 4b relative to the depth p is long. With reference to FIG. 2a, let d1 be the distance separating the bottom of the second slot 3b from the part of the end of the branch 4a which defines it which is the furthest away from it and let d2 be the distance separating the bottom of the third slot 3c from the part of the end of the branch which defines it which is the furthest away from it, when the anchor 1 is in the rest position. The suture anchor 1 is designed such that each of the ratios d1/p and d2/p is greater than or equal to 0.3. In the anchor 1 shown in the figures, d1=d2=1.8 mm and p=2.75 mm. The ratios d1/p and d2/p are thus both approximately equal to 0.65.

The suture anchor 1 is intended to be anchored in the distal phalanx P3 of a finger of the hand. It must have the highest possible expansion potential which is translated by a low ratio e2/e1, i.e. a low reduction coefficient “k”.

The relative proportions of the previously defined values d1, d2 and p are used in obtaining an advantageous ratio between the span perpendicular to the axis x-x′ in the rest position (e1) and the span perpendicular to the axis x-x′ in the extreme folded position (e2).

The front and rear surfaces of the suture anchor 1 are typically planar and in parallel with a single plane P1 including the axis x-x′. The suture anchor 1 typically has a plane of orthogonal symmetry P2 normal to the plane P1 and likewise including the axis x-x′. These planes P1 and P2 are shown in FIG. 1a.

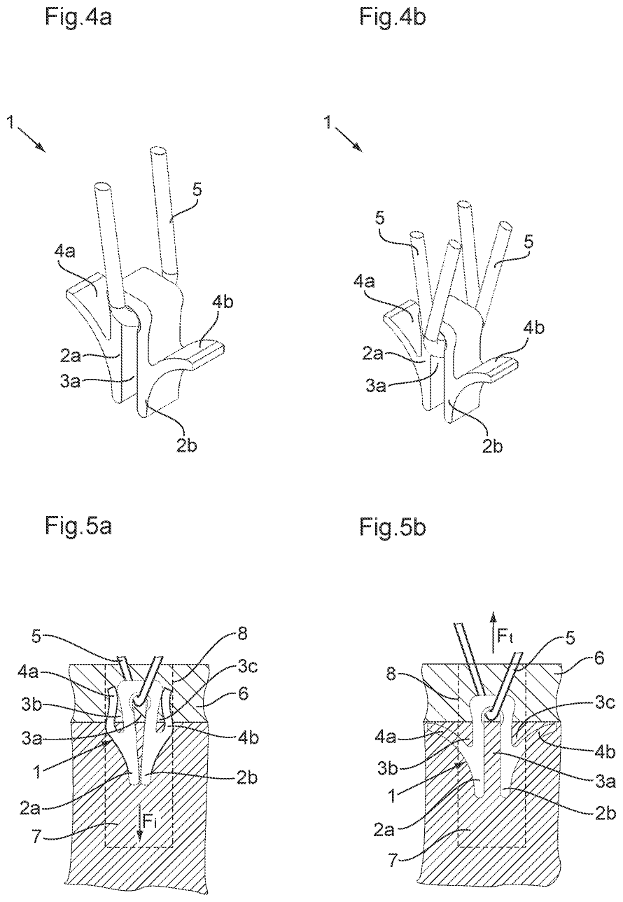

In order to ref ix the damaged tendons or ligaments in their place on the phalanx P3, the suture anchor 1 is typically anchored in this phalanx, beneath the cortical bone. It bears one or more suture threads 5 for suturing the damaged tissue, tendon or ligament. FIG. 4a shows the anchor 1 in the expanded position with a suture thread in position in the first slot 3a. Given that the first slot 3a has an open contour in the rest position, in this position the surgeon can himself select and position the suture thread 5 he requires and do so without the use of a thread guide. The first slot 3a is also sufficiently deep to be able to accommodate several suture threads 5 if necessary, as shown in FIG. 4b.

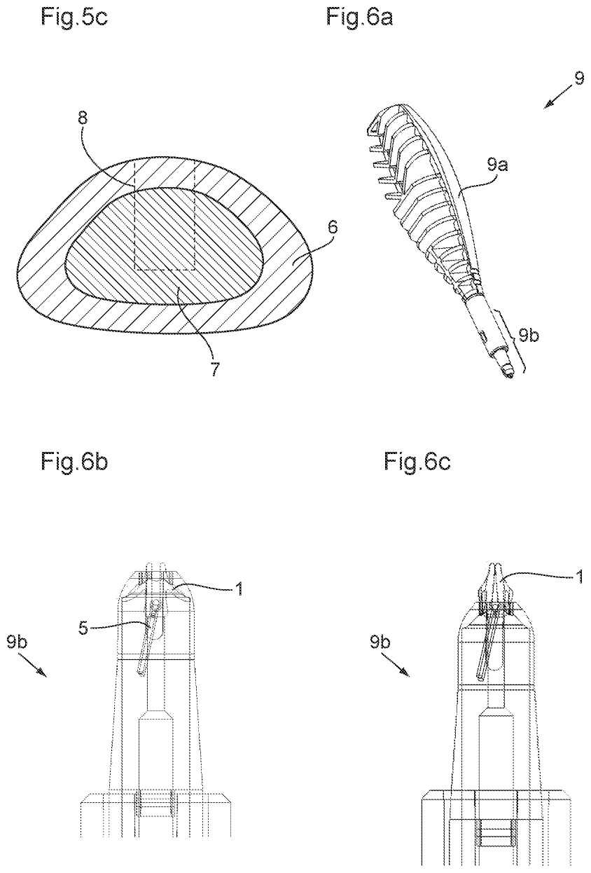

In practice, the suture anchor 1 is intended to be inserted into a hole 8 passing all the way through the dorsal cortical part 6 of the phalanx P3 and extending in part into the spongy bone 7 as shown in FIGS. 5a, 5b and 5c.

The hole 8 is typically a cylindrical piercing, of which the diameter is the smallest diameter allowing passage of the anchor 1 when it is in the extreme folded position. This diameter is typically equal to the maximum span e2 of the anchor 1 in the extreme folded position, i.e. 2 mm. It is the longest length separating two parts of the anchor 1 in the extreme folded position, perpendicular to the axis x-x′ (in the top view). The diameter of this piercing is likewise sufficiently narrow to prevent the anchor 1 from exiting the hole 8 once expanded in the bone.

The suture anchor 1 is intended to be inserted with its lower part (side x of the axis x-x′) at the front in the direction of an insertion force Fᵢ oriented in the extension of the hole 8, i.e. following the axis of revolution of the cylindrical piercing as shown in FIG. 5a. Its branches 4a, 4b are intended to pass completely through the thickness of the cortical bone 6 and then to expand into the spongy bone 7 under the effect of the elastic restoring forces being exerted on the arms 2a, 2b and on the branches 4a, 4b and tending to bring them back to their rest position, as shown in FIG. 5b.

In order to permit firm anchoring without risking damage to the cortical bone, the anchor 1 must be designed such that its arms and branches are able to expand sufficiently, from the folded insertion position thereof, ideally almost completely or even completely, when in the spongy bone 7 but so that they are unable to expand, even partially, when in the cortical bone 6.

Within the scope of the invention, it is considered that the assembly comprising the arms 2a, 2b and branches 4a, 4b of the anchor 1 is able to expand in the spongy bone 7 if this assembly is able to expand from a first position corresponding to its folded insertion position to a sufficiently expanded second position when embedded in any material in which the compression failure pressure is less than or equal to 10 MPa, preferably less than or equal to 15 MPa.

The “folded insertion position” is understood to be a position in which the span of the assembly comprising the arms 2a, 2b and branches 4a, 4b of the anchor 1 perpendicular to the axis x-x′ is reduced by at least 30%, preferably at least 40%, even more preferably at least 45%, relative to its span perpendicular to the axis x-x′ in the rest position, and “sufficiently expanded position” is understood to be a position in which the span of this assembly perpendicular to the axis x-x′ is equal to at least 75%, preferably at least 80%, preferably at least 85%, preferably at least 90%, even more preferably at least 95%, or even 100%, of its span perpendicular to the axis x-x′ in its expanded (rest) position.

Within the scope of the invention, it is likewise considered that the anchor 1 cannot expand even partially in the cortical bone 6 if the assembly comprising the arms 2a, 2b and branches 4a, 4b thereof is incapable of expanding, even partially, when it is embedded in any material in which the compression failure pressure is greater than or equal to 200 MPa, preferably greater than or equal to 100 MPa, preferably greater than or equal to 50 MPa.

Once the anchor 1 is in place in the bone and the suture is formed, the suture thread 5 exerts a traction force Ft on the anchor 1. The branches 4a, 4b expand in the spongy bone 7 and the parts of the branches 4a, 4b which, in the rest position, are oriented opposite to the axis x-x′ typically come to abut against the inside of the cortical part 6 of the phalanx P3, thereby preventing the anchor 1 from exiting same. This is shown in FIG. 5b.

In order to insert the suture anchor 1 into the hole 8, the surgeon typically uses forceps or an instrument 9 as shown in FIG. 6a.

Such an instrument 9 comprises a handle 9a and an active part 9b. The active part 9b comprises a housing in which the anchor 1 may be positioned and held in the expanded position (cf. FIG. 6b). A pusher allows a force to be exerted on the upper part of the anchor 1 to fold it, typically to the extreme folded position, introducing a force into an opening with suitable dimensions (cf. FIG. 6c).

The suture anchor 1 is particularly suitable for being used in a distal phalanx P3 of a finger of the hand but can be used in other bones, its dimensions able to be adapted as need be.

A method for placing an orthopaedic suture anchor 1 as previously defined typically comprises the following steps:
producing a piercing 8 passing all the way through the cortical part 6 of a bone and extending into the spongy part 7 of said bone;
positioning one or more suture threads 5 in the first slot 3a of said suture anchor 1, this typically being able to be performed by the surgeon, for example with the suture anchor 1 held in the expanded position in an instrument such as the previously described instrument 9;
bringing the suture anchor 1 into a folded insertion position, i.e. reducing the span of the assembly comprising the first 2a and second 2b arms thereof and the first 4a and second 4b branches thereof perpendicular to the axis x-x′ by at least 30% with respect to said span in the rest position, on the one hand by elastically bringing each of the branches 2a, 2b closer to the arm 4a, 4b which bears it, and on the other hand by elastically bringing said first 2a and second 2b arms closer together, this typically being able to be performed by the surgeon by exerting a pressure on the branches 4a, 4b using forceps or using an instrument such as the previously described instrument 9;
inserting the assembly comprising said first 2a and second 2b arms and said first 4a and second 4b branches through this piercing 8 and keeping it in said folded insertion position;
ceasing to keep the span of said assembly in said folded insertion position, typically by relaxing the pressure exerted on the branches 4a, 4b thereof such that said assembly expands at least partially in the spongy part 7 of said bone; and
suturing a damaged tissue such as a ligament or tendon with the suture thread(s) 5.

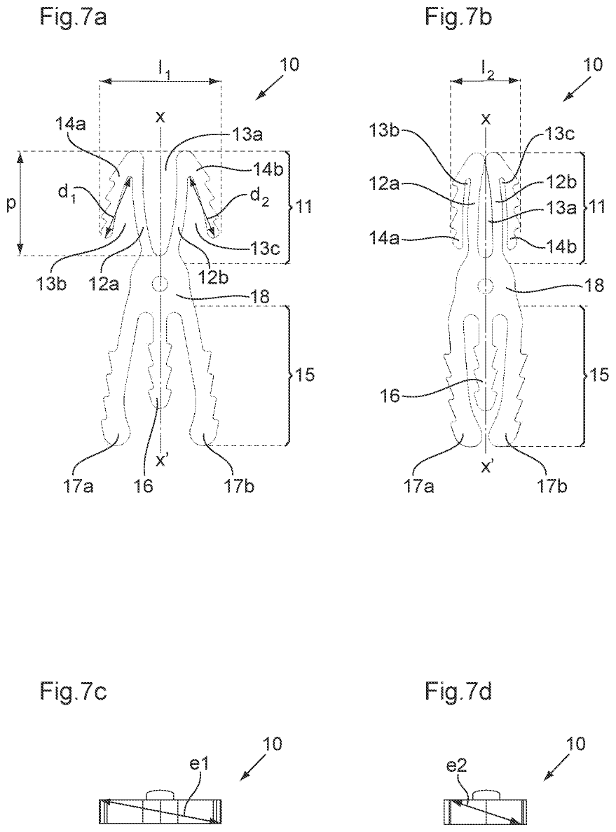

With reference to FIGS. 7a to 10, a bone anchoring device in accordance with a second embodiment of the invention is an implant 10 for arthrodesis, typically for a distal interphalangeal articulation.

The implant 10 comprises a first anchoring part 11 intended to be anchored in a distal phalanx P3 of a finger of the hand and a second anchoring part 15 intended to be anchored in the middle phalanx P2 biologically articulated to said distal phalanx P3 so as to form a bone bridge between these two phalanges.

These first 11 and second 15 anchoring parts are connected via a central rigid portion 18.

The first anchoring part 11 has a structure very similar to that of the suture anchor 1 in accordance with the first embodiment of the invention. It comprises a first 12a and a second 12b arm as well as first 14a and second 14b branches.

The first 12a and second 2b arms are typically arranged in a U- or V-shape and define a first slot 13a therebetween. They respectively bear said first 14a and second 14b branches which extend outside of said first slot 13a.

This first slot 13a has a depth of length p and extends along an axis x-x′, x corresponding to the upper part and x′ corresponding to the lower part of the implant 10, as shown in FIGS. 7a and 7b.

The first branch 14a is arranged so as to define, with the arm 12a which bears it, a second slot 13b. Similarly, the second branch 14b is arranged so as to define, with the arm 12b which bears it, a third slot 13c.

The implant 10 is typically in one piece.

In its rest position, i.e. in the position in which no external force is applied thereon, the first anchoring part 11 of the implant 10 is in an “expanded” position. This rest position is shown in FIG. 7a. In this rest position, the span (maximum or overall) of the first anchoring part 11, perpendicular to the axis x-x′, this span corresponding to the span of the assembly comprising said first 12a and second 12b arms and said first 14a and second 14b branches perpendicular to the axis x-x′, corresponds to a value e1, shown in FIG. 7c.

From this rest position, this span can be reduced on the one hand by bringing the two arms 12a, 12b closer together by elastic deformation and on the other hand by bringing each of the branches 14a, 14b closer to the arm 12a, 12b which bears it, and thus to the axis x-x′, as shown in FIG. 7b.

As for the suture anchor 1, the “extreme folded position” is the name given to the position in which the span (maximum or overall) of the first anchoring part 11 of the implant 10 perpendicular to the axis x-x′ is minimum. In this position, the ends of the two arms 12a, 12b touch each other such that the contour of the first slot 13a is closed and each of the branches 14a, 14b is folded to the maximum extent against the arm 12a, 12b which bears it. This position is shown in FIG. 7b and corresponds to the ideal insertion position of the first anchoring part 11 of the implant 10 into a distal phalanx P3 of a finger. In this position, the span of the first anchoring part 11 perpendicular to the axis x-x′ corresponds to a value e2<e1 , shown in FIG. 7d. The reduction coefficient k of this span corresponds to the ratio e2/e1 and is typically less than or equal to 0.7.

The first anchoring part 11 of the implant 10 shown in the figures has a span e1, at rest, of 6.1 mm and a span e2, in the extreme folded position, of 3.3 mm, the reduction coefficient is thus approximately k=0.54. A low reduction coefficient translates into a large expansion potential. Such properties are made possible owing to the use of a superelastic material such as a 55% nickel/45% titanium alloy for manufacturing the implant 10.

The span e1 is slightly greater than the width l1=6 mm of the first anchoring part 11 in the expanded position (FIGS. 7a and 7c). Similarly, the span e2 is slightly greater than the width l2=3.1 mm of the first anchoring part 11 in the extreme folded position (FIGS. 7b and 7d).

The arms 12a, 12b and branches 14a, 14b of the first anchoring part 11 of the implant 10 are arranged such that the orthogonal projection on the axis x-x′ of the bottom of each of the second 13b and third 13c slots is located in said first slot 13a at a distance from the bottom of this slot of at least 10%, preferably at least 20%, preferably at least 30%, preferably at least 40%, preferably at least 50%, even more preferably at least 75%, of the depth p. In the example shown in FIGS. 7a to 10, the orthogonal projection on the axis x-x′ of the bottom of each of the second 3b and third 3c slots is located in said first slot 3a at a distance from the bottom of this slot of about 84%.

Furthermore, the arms 12a, 12b and branches 14a, 14b of the first anchoring part 11 of the implant 10 are arranged such that, in the rest position, the orthogonal projection on the axis x-x′ of each of said first 14a and second 14b branches is located fully in said first slot 13a (cf. FIG. 7a). As a variant, it would be possible for only one part of the length of the orthogonal projection of each of the branches 14a, 14b on the axis x-x′, typically at least 70% of this length, to be located in the first slot 13a in the rest position.

As for the suture anchor 1, the length of the branches 14a, 14b of the implant 10 relative to the depth p is long. With reference to FIG. 7a, let d1 be the distance separating the bottom of the second slot 13b from the part of the end of the branch 14a which defines it which is the furthest away from it and let d2 be the distance separating the bottom of the third slot 13c from the part of the end of the branch 14b which defines it which is the furthest away from it, when the first anchoring part 11 is in the rest position. The implant 10 is designed such that each of the ratios d1/p and d2/p is greater than or equal to 0.3. With respect to the first anchoring part 11 of the implant 10, d1=d2=3.45 mm and p=5.5 mm such that d1/p and d2/p are both approximately equal to 0.63.

As previously stated, the first anchoring part 11 of the implant 10 is intended to be anchored in the distal phalanx P3 of a finger of the hand. Given the small dimensions of such a bone, the piercing for the insertion of the anchoring part 11 into the phalanx P3 must have the smallest possible diameter. In fact, the preservation of the bone is important for the mechanical strength of the phalanx P3 as well as to maximise the bone surface in contact between the phalanges P2 and P3 for the bone fusion thereof. Despite this constraint, the implant 10 must allow anchoring which is as strong as possible. The first anchoring part 11 must thus have a high expansion potential which is translated by a low ratio e2/e1 and thus a low reduction coefficient k.

The relative proportions of the previously defined values d1, d2 and p are used in obtaining an advantageous ratio between the span perpendicular to the axis x-x′ in the rest position (e1) and the span perpendicular to the axis x-x′ in the extreme folded position (e2).

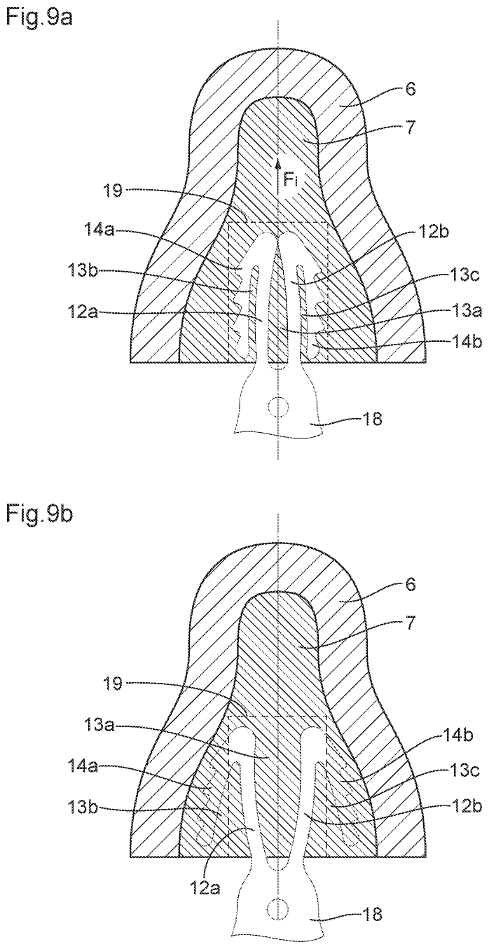

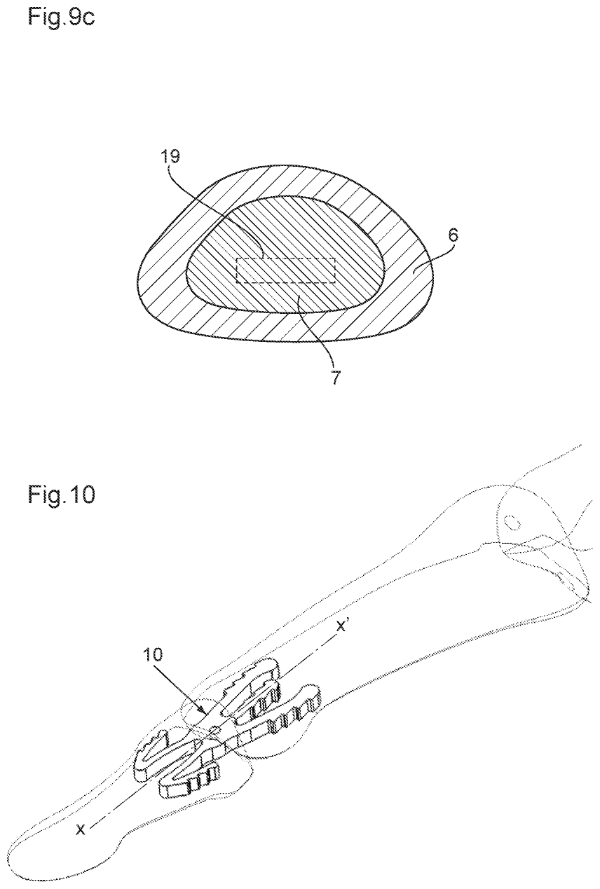

In practice, the first anchoring part 11 will be inserted into a first hole 19 extending in the spongy bone part 7 of the phalanx P3, as shown in FIGS. 9a, 9b and 9c.

In order to produce such a hole 19, the phalanx P3 is squared-off at its junction with the phalanx P2 and then the hole 19 is produced in the spongy bone 7. FIG. 9a illustrates a longitudinal section of the phalanx P3 after squaring-off and production of the hole 19. The hole 19 is typically a sized blind hole, produced using a special rasp. Its dimensions are adapted to the dimensions of the first anchoring part 11.

The first anchoring part 11 is intended to be inserted with its upper part (side x′ of the axis x-x′) at the front in the direction of an insertion force Fi oriented in the extension of the first hole 19, as shown in FIG. 9a. The branches 14a, 14b are intended to be inserted fully into said first hole 8 and then to expand into the spongy bone 7 under the effect of the elastic restoring forces being exerted on the arms 12a, 12b and branches 14a, 14b and tending to bring them back to their rest position, as shown in FIG. 9b.

In order to permit firm anchoring without risking damage to the cortical bone 6, the first anchoring part 11 must be designed such that its arms 12a, 12b and branches 14a, 14b are able to expand sufficiently, from the folded insertion position thereof, ideally almost completely or even completely, when in the spongy bone 7 but so that they are unable to expand, even partially, when in the cortical bone 6.

As for the first embodiment of the invention, it is considered that the assembly comprising the arms 12a, 12b and branches 14a, 14b of the first anchoring part 11 of the implant 10 is able to expand in the spongy bone 7 if this assembly is able to expand from a first position corresponding to its folded insertion position to a sufficiently expanded second position when embedded in any material in which the compression failure pressure is less than or equal to 10 MPa, preferably less than or equal to 15 MPa.

The “folded insertion position” is understood to be a position in which the span of the assembly comprising the arms 12a, 12b and branches 14a, 14b of the first anchoring part 11 perpendicular to the axis x-x′ is reduced by at least 30%, preferably at least 40%, even more preferably at least 45%, relative to its span perpendicular to the axis x-x′ in the rest position, and “sufficiently expanded position” is understood to be a position in which the span of this assembly perpendicular to the axis x-x′ is equal to at least 75%, preferably at least 80%, preferably at least 85%, preferably at least 90%, even more preferably at least 95%, or even 100%, of its span perpendicular to the axis x-x′ in its expanded (rest) position.

Within the scope of the invention, it is likewise considered that the first anchoring part 11 of the implant 10 cannot expand even partially in the cortical bone 6 if the assembly comprising the arms 12a, 12b and branches 14a, 14b thereof is incapable of expanding, even partially, when it is embedded in any material in which the compression failure pressure is greater than 200 MPa, preferably greater than or equal to 100 MPa, preferably greater than or equal to 50 MPa.

Owing to the structure of the arms 12a, 12b and branches 14a, 14b thereof, the first anchoring part 11 of the implant 10 is hourglass-shaped. This shape is particularly adapted to the shape of the distal phalanx P3 of the fingers of the hand, as shown in FIG. 10. Owing to this shape, the sides of the branches 14a, 14b facing away from the axis x-x′ can come into abutment against the inside of the cortical part 6 of the phalanx, creating non-punctiform support zones, typically distributed over the entire length of the branches 14a, 14b, which avoids damage to the bone. The branches 14a, 14b are typically provided with notches to improve the anchoring thereof in the phalanx P3.

The front and rear surfaces of the first 12a and second 12b arms and the first 14a and second 14b branches of the first anchoring part 11 are planar and in parallel with a single first plane including the axis x-x′.

Since the implant 10 is a intramedullary implant for DIP arthrodesis, its second anchoring part 15 has a different shape from the first anchoring part 11, this shape being particularly adapted to the shape of the phalanges P2.

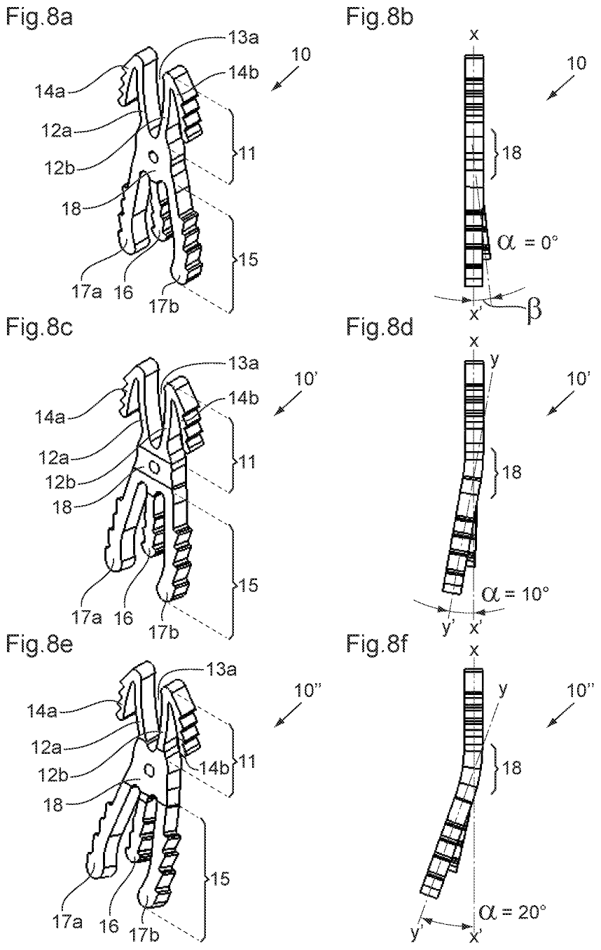

The second anchoring part 15 typically comprises three arms 16, 17a, 17b arranged such that, in the rest position, two 17a, 17b of said arms, called main arms, define the span (maximum or overall) of said second anchoring part 15 perpendicular to an axis y-y′. In the implant 10 shown in FIGS. 7a to 8b, the axis y-y′ merged with the axis x-x′.

Likewise for the second anchoring part 15, it is important that its span perpendicular to the axis y-y′ is smaller in the extreme folded state than in the rest state, i.e. it has a high expansion potential between the folded position in which it is inserted into the bone and the position it will assume once expanded.

The second anchoring part 15 extends for the most part in a second plane including the axis y-y′.

As a variant, the axis y-y′ can form an angle α with the axis x-x′, α typically being between 0° and 40°, preferably between 0° and 25°, even more preferably between 0° and 20°. In the example shown in FIGS. 7a to 8b, α=0°.

FIGS. 8c to 8f show two variants of implants belonging to a range of implants in accordance with the invention. In a first of these variants (implant 10′ shown in FIGS. 8c and 8d), the angle α is 10°. In a second of these variants (implant 10″ shown in FIGS. 8e and 8f), the angle α is 20°.

The variants of implants 10, 10′, 10″ form a range of implants from which the surgeon can choose in order to adapt the surgery based on the joint affected and the wishes of the patient. This is particularly important in the case of arthrodesis on a DIP joint. In fact, the angle between the distal and middle phalanges is crucial in the gripping function of the hand.

It is the central rigid portion 18 of each of the implants 10, 10′, 10″ which determines the angle α between said first and second planes.

Advantageously, the central rigid portion 18 of the implant 10 comprises a central piercing. This allows the implant 10, 10′, 10″ to be kept in position via a Kirschner pin during the surgery, this pin being removed at the end of surgery.

The third 16 of the arms of the second anchoring part 15, called additional arm, extends in a third plane forming an angle β of approximately 10° with said second plane. Said three arms 16, 17a, 17b form a tripod. This can be seen in FIG. 8a. As a variant, the angle β can vary. It is typically between 7° and 15°, preferably between 10° and 15°.

The main arms 17a, 17b of the second anchoring part 15 are typically provided with notches. The additional arm 16 of the second anchoring part 15 is preferably likewise provided with notches. These notches all aim to improve the anchoring of said arms 16, 17a, 17b in the phalanx P2.

The arrangement of the three arms 16, 17a, 17b in the shape of a tripod permits good anchoring. Such an arrangement is particularly suitable for a middle phalanx P2 of a finger of the hand. It provides dorsal-palmar stability to the implant 10, 10′, 10″ and prevents break-down of the dorsal cortex.

In practice, the second anchoring part 15 is inserted into a second hole produced in the cortical part of the phalanx P2.

As for the first hole 19, this second hole is a sized blind hole, typically produced using a special rasp after squaring-off of the phalanx P2, and the dimensions of which are adapted to the dimensions of the second anchoring part 15.

The main feature of the arthrodesis implants 10, 10′, 10″ described above resides in the fact that the first anchoring part 11 thereof has a double expansion of its arms 12a, 12b and also of its branches 14a, 14b.

In each of the arthrodesis implants 10, 10′, 10″ shown in FIGS. 7a to 10, the first 11 and second 15 anchoring parts are different from one another, each have a shape particularly adapted to the phalanx with which it cooperates for the DIP arthrodesis. However, to the extent that such implants 10, 10′, 10″ could be used in other bones, a person skilled in the art could adapt the shapes and dimensions of the anchoring parts 11, 15 as a function of the bones for which arthrodesis would be envisaged. For example, they could both be identical to the first anchoring part 11.

A method for placing an implant 10, 10′, 10″ as previously defined typically comprises the following steps:
i. producing a first sized hole 19 in the spongy part 7 of a first bone of a joint between two bones to be treated and a second sized hole in the spongy part of the second bone of said joint;
ii. anchoring the second anchoring part 15 of the implant 10 in said second hole by performing the following steps:
   bringing the second anchoring part 15 of the implant 10 into a folded insertion position, i.e. reducing the span of said second anchoring part 15 perpendicular to the axis y-y′, this typically being able to be performed by the surgeon by exerting a pressure on the main arms 17a, 17b for example using forceps or using an insertion instrument;
   inserting the second anchoring part 15 into the second hole, keeping it in said folded insertion position; and
   ceasing to keep the second anchoring part 15 in said folded insertion position, typically by relaxing the pressure exerted on the arms 17a, 17b;
iii. anchoring the first anchoring part 11 of the implant 10 in said first hole by performing the following steps:
   bringing the first anchoring part 11 of the implant 10 into a folded insertion position, i.e. reducing the span of the assembly comprising said first 12a and second 12b arms and said first 14a and second 14b branches perpendicular to the axis x-x′ by at least 30% with respect to said span in the rest position, on the one hand by elastically bringing each of the branches 14a, 14b closer to the arm 12a, 12b which bears it, and on the other hand by elastically bringing said first 12a and second 12b arms closer together, this typically being able to be performed by the surgeon by exerting a pressure on the branches 14a, 14b for example using forceps;
   inserting the assembly comprising said first 12a and second 12b arms and said first 14a and second 14b branches of said first anchoring part 11 into said first hole 19, keeping it in said folded insertion position;
   ceasing to keep said assembly in said folded insertion position, typically by relaxing the pressure exerted on the branches 14a, 14b such that said assembly expands at least partially in the spongy part 7 of said first bone.

As a variant, step iii can be performed prior to step ii.

Preferably, a step preliminary to step i consists of squaring-off the joint surface of each of the first and second bones of said joint to be treated.

It will be clear to a person skilled in the art that the present invention is in no way limited to the embodiments presented above and illustrated in the figures.

It is clear that the shape of the arms and branches of the anchor or of the first anchoring part of the implant in accordance with the invention can vary in an infinite number of ways so long as the function thereof is ensured.

It is likewise very feasible to produce a bone anchoring device in accordance with the invention other than a suture anchor or implant for arthrodesis, for example an arthroplasty rod or interference screw.

The bone anchoring device in accordance with the invention has the advantage of having a high expansion potential, i.e. it has a folded position in which its span is very narrow and an expanded position in which its span can be much wider. Therefore, in the folded position it can be inserted into holes with an extremely small diameter, whilst allowing strong anchoring by expanding in the bone. Typically, for a given span in the extreme folded state, an expansion of the assembly comprising the arms and branches of the anchoring part much greater than that of the anchoring devices of the prior art is obtained. This is of much interest, in particular for bone anchoring devices used in small bones of which the dimensions and fragility limit the size of the piercing.

1. Bone anchoring device (1; 10) comprising a first (2a; 12a) and a second (2b; 12b) arm defining therebetween a first slot (3a; 13a) of depth p extending along an axis x-x′, said first (2a; 12a) and second (2b; 12b) arms respectively bearing a first (4a; 14a) and a second (4b; 14b) branch extending outside of said first slot (3a; 13a), said first branch (4a; 14a) being arranged so as to define with the arm (2a, 12a) which bears the first branch a second slot (3b; 13b) and said second branch (4b; 14b) being arranged so as to define with the arm (2b; 12b) which bears the second branch a third slot (3c; 13c), said device (1; 10) being arranged such that, in the rest position:
the orthogonal projection on the axis (x-x′) of the bottom of each of the second (3b; 13b) and third (3c; 13c) slots is located in said first slot (3a; 13a) at a distance from the bottom of this slot of at least 10%, of the depth p; and
the distances d1 separating the bottom of said second slot (3b, 13b) from the part of the end of the branch (4a; 14a) which defines it which is the furthest away from it, and d2 separating the bottom of said third slot (3c; 13c) from the part of the end of the branch (4b; 14b) which defines it which is the furthest away from it, are such that each of the ratios d1/p and d2/p is greater than or equal to 0.3;
said device (1; 10) being able to reduce the span of the assembly comprising said first (2a; 12a) and second (2b; 12b) arms and said first (4a; 14a) and second (4b; 14b) branches perpendicular to the axis (x-x′) from said rest position, both by elastically bringing each of the branches (4a, 4b; 14a, 14b) closer to the arm (2a, 2b; 12a, 12b) which bears each said branch, and also by elastically bringing said first (2a; 12a) and second (2b; 12b) arms closer together for the insertion thereof, at least in part, into a bone.

2. The bone anchoring device (1; 10) as claimed in claim 1, wherein the span of the assembly comprising said first (2a; 12a) and second (2b; 12b) arms and said first (4a; 14a) and second (4b; 14b) branches perpendicular to the axis x-x′ can, when said elements are elastically brought closer together, be reduced by at least 30% with respect to said span in the rest position.

3. The bone anchoring device (1; 10) as claimed in claim 1, wherein the device is designed such that the assembly comprising the arms (2a, 2b; 12a, 12b) and branches (4a, 4b; 14a, 14b) is able to expand from a first folded position in which [[its]] the assembly's span perpendicular to the axis x-x′ is less than or equal to 0.7 times the assembly's span perpendicular to the axis x-x′ in the rest position, to a second position in which the assembly's span perpendicular to the axis x-x′ is greater than or equal to 0.75 times the assembly's span perpendicular to the axis x-x′ in the rest position, into the materials in which the compression failure pressure is less than or equal to 10 MPa, whilst being incapable of expanding even partially into materials in which the compression failure pressure is greater than or equal to 200 MPa.

4. The bone anchoring device (1; 10) as claimed in claim 1, comprising a superelastic material.

5. The bone anchoring device (1; 10) as claimed in claim 1, wherein in said rest position the orthogonal projection on the axis x-x′ of each of said first (4a; 14a) and second (4b; 14b) branches is located fully or almost fully in said first slot (3a; 13a).

6. The bone anchoring device (1; 10) as claimed in claim 1, wherein the bone anchoring device is in one piece.

7. The bone anchoring device as claimed in claim 1, wherein the bone anchoring device is a suture anchor (1) and wherein said first slot (3a) is intended to receive one or more suture threads (5) to suture a tissue.

8. The bone anchoring device (10) as claimed in claim 1, further comprising a first anchoring part (11) comprising said first (12a) and second (12b) arms and said first (14a) and second (14b) branches and intended to be anchored in a first bone, and a second anchoring part (15), made fixedly attached to the first part, and intended to be anchored in a second bone, the shape of said second anchoring part (15) being able to be identical to or different from that of said first anchoring part (11).

9. The bone anchoring device as claimed in claim 8, wherein said first anchoring part (11) extends in a first plane and wherein said second anchoring part (15) extends mostly in a second plane forming an angle α, typically between 0° and 40°, with said first plane, said central rigid portion (18) determining the angle α between said first and second planes.

10. The bone anchoring device as claimed in claim 9, wherein said second anchoring part (15) comprises three arms (16a, 16b, 17) arranged such that, in the rest position, two (16a, 16b) of said arms, called main arms, define the span of said second anchoring part perpendicular to an axis (y-y′) forming said angle α with the axis (x-x′) and extend in said second plane including this axis (y-y′) and the third (17) of said arms, called additional arm, extends in a third plane forming an angle β with said second plane.

11. The bone anchoring device as claimed in claim 10, wherein said main arms (16a, 16b) of said second anchoring part (15) are provided with notches.

12. The bone anchoring device (1) as claimed in claim 1, wherein said bone or said first and second bones are selected from a phalanx of the foot or of the hand.

13. Kit comprising a bone anchoring device as claimed in any claim 1 and an instrument able to bear said device and keep said device at least in a position in which the span perpendicular to the axis x-x′ of the assembly comprising said first and second arms and said first and second branches is reduced by at least 30% with respect to said span in the rest position.

14. The bone anchoring device of claim 1, wherein:
the orthogonal projection on the axis of the bottom of each of the second and third slots is located in said first slot at a distance from the bottom of this slot of at least 50% of the depth p; and
each of the ratios d1/p and d2/p is greater than or equal to 0.5.

15. The bone anchoring device (1; 10) as claimed in claim 1, wherein the device is designed such that the assembly comprising the arms (2a, 2b; 12a, 12b) and branches (4a, 4b; 14a, 14b) is able to expand from a first folded position in which the assembly's span perpendicular to the axis x-x′ is less than or equal to 0.55 times the assembly's span perpendicular to the axis x-x′ in the rest position, to a second position in which the assembly's span perpendicular to the axis x-x′ is greater than or equal to 0.95 times the assembly's span perpendicular to the axis x-x′ in the rest position, into the materials in which the compression failure pressure is less than or equal to 15 MPa, whilst being incapable of expanding even partially into materials in which the compression failure pressure is greater than or equal to 50 MPa.

16. The bone anchoring device (1; 10) as claimed in claim 4, wherein the superelastic material is a nickel/titanium alloy.

17. The bone anchoring device as claimed in claim 9, wherein said angle α, measures between 0° and 25°.

18. The bone anchoring device as claimed in claim 11, wherein said additional arm (17) of said second anchoring part (15) is likewise provided with notches, said notches being intended to be in contact with said second bone.

19. The bone anchoring device (1; 10) as claimed in claim 2, wherein the device is designed such that the assembly comprising the arms (2a, 2b; 12a, 12b) and branches (4a, 4b; 14a, 14b) is able to expand from a first folded position in which the assembly's span perpendicular to the axis x-x′ is less than or equal to 0.7 times the assembly's span perpendicular to the axis x-x′ in the rest position, to a second position in which the assembly's span perpendicular to the axis x-x′ is greater than or equal to 0.75 times the assembly's span perpendicular to the axis x-x′ in the rest position, into the materials in which the compression failure pressure is less than or equal to 10 MPa, whilst being incapable of expanding even partially into materials in which the compression failure pressure is greater than or equal to 200 MPa.

20. The bone anchoring device (1; 10) as claimed in claim 2, comprising a superelastic material.
